(19) United States
(12) Patent Application Publication
(10) Pub. No.: US 2021/0300876 A1
(43) Pub. Date: Sep. 30, 2021

(54) DIENE GEMINI POLYMERIZABLE SURFACTANTS WITH MIXED CIS AND TRANS ISOMERS THAT FORM BICONTINUOUS CUBIC PHASES

(71) Applicant: TDA Research, Inc, Wheat Ridge, CO (US)

(72) Inventors: Rhia M Martin, Arvada, CO (US); Douglas L. Gin, Longmont, CO (US); Richard D Noble, Boulder, CO (US); Vinh The Nguyen, Arvada, CO (US); Brian J Elliott, Superior, CO (US)

(21) Appl. No.: 17/259,467

(22) PCT Filed: Jul. 9, 2019
(86) PCT No.: PCT/US19/41054

§ 371 Date: Jan. 11, 2021

Related U.S. Application Data
(60) Provisional application No. 62/695,382, filed on Jul. 9, 2018.

(51) Int. Cl. C07D 233/58; C09K 19/52

(52) U.S. Cl. CPC C07D 233/58; C09K 2019/528; C09K 19/52

(57) ABSTRACT

A polymerizable Gemini surfactant based on tail groups with mixed isomer dienes. The Gemini surfactants may be produced having imidazolium head groups and diene tail groups with a near-equal abundance of the “E” and “Z” isomers. These compounds are lyotropic liquid crystals that can form bicontinuous cubic phases by self-assembly.

### CROSS REFERENCE TO RELATED APPLICATIONS

The present application claims the benefit of the provisional application No. 62/695,382 filed Jul. 9, 2018 (titled DIENE GEMINI POLYMERIZABLE SURFACTANTS WITH MIXED CIS AND TRANS ISOMERS THAT FORM BICONTINUOUS CUBIC PHASES, by Rhia M Martin, Douglas L Gin, Richard D Noble, Vinh The Nguyen and Brian J Elliott, attorney docket number 18-2), which is incorporated by reference herein.

### STATEMENT REGARDING U.S. GOVERNMENT SUPPORT

This invention was made in part using U.S. government funding through the U.S. Department of Energy SBIR Phase II contract # DE-SC0015205. The government has certain rights in this invention.

### FIELD OF THE INVENTION

The present invention relates generally to polymerizable surfactant compounds that contain a diene functional group having near-equal proportions of the cis and trans conformations. The present invention further relates to cubic-phase forming diene “Gemini” polymerizable surfactants, in which the abundance of either the cis or the trans conformation does not exceed 60% of the total diene groups (i.e. each isomer is present at an abundance of from 40 to 60%).

### BACKGROUND OF THE INVENTION

Liquid crystal phase-forming polymerizable surfactants are useful for producing self-assembled nanostructured polymers and nanoporous items of manufacture including membranes for desalination, air purification or as battery separator materials (U.S. Pat. No. 8,163,204 B2 and U.S. Pat. No. 14/234,362). Polymerizable surfactants are compounds containing one or more charged or polar head group(s) and one or more hydrophobic tails group(s), wherein the tail group(s) contain a polymerizable functional group. In some cases this polymerizable group is an acrylate or a methacrylate, however, the resulting polymers that result from a free radical polymerization of acrylate or methacrylate monomers contain ester linkages, which are not as chemically or electrolytically stable as other polymer materials that do not have ester linkages. For example, diene polymerizable groups form polymer with no ester linkages and are more stable.

Although there are numerous lyotropic liquid crystalline phases, particularly advantageous phases for forming membranes or nanoporous polymer materials are cubic phases. Bicontinuous cubic phases of polymerizable surfactants (there are type-I and type-II bicontinuous cubic geometries, for example) contain a 3-dimensional network of interconnecting pores that enable superior diffusion of solutes that are smaller than the pore diameter (U.S. Pat. No. 14/234,362). Cubic phases do not suffer from the requirement to align nanopores in a specific orientation for diffusion of small molecules across a membrane or through other polymer films such as battery separators.

One particularly useful type of polymerizable surfactant that forms the bicontinuous cubic phase is a “Gemini” surfactant having two ionic headgroups, a headgroup spacer groups and two tail groups, each attached to one of the headgroups.

The prior art teaches that it is important to have polymerizable surfactants with diene tail groups having nearly all trans (or E) conformations. The trans conformation is the more “straight” conformation. The cis (or Z) conformation is avoided. Forming the cubic phase is difficult because it has been found to be very sensitive to variations in surfactant geometry and it has a very narrow concentration window for forming the cubic phase. Hoag et al. (Macromolecules 33, No 23, 2000, pp 8554) teach a lengthy chemical synthesis for producing a Gemini-diene polymerizable surfactant with greater than 95% purity of the trans (E) conformation. A Peterson elimination under acidic conditions affords the nearly pure isomer. Although performing the Peterson elimination under basic conditions affords greater than 90% of the cis (Z) isomer, the cis isomer is never used in any of the prior art references to form nanoporous or cubic phase materials. There is a general bias implied or understood that the trans isomer will lead to the successful formation of the bicontinuous cubic phase, and it must be in a highly pure state. All prior art references also illustrate the chemical structure with the trans isomer and teach this preference of the trans isomer for successful cubic phase forming behavior (including forming cubic pores for water desalination). Obtaining this pure isomer requires a lengthy and expensive synthesis and the use of a “Matteson's reagent” as an intermediate step. Several imidazolium-based polymerizable LLC surfactants are described in U.S. Pat. No. 7,931,824 B2, which is incorporated by reference herein in its entirety for its description of exemplary polymerizable LLC surfactants.

The above prior art relating to Gemini-diene polymerizable surfactants suffer from at least one of the following limitations: they require lengthy synthesis to produce a nearly pure isomer of the diene, they require a Matteson's Reagent as an intermediate step, they are difficult to make, or they do not allow for mixed isomers of the diene. The prior art also suffers from the teaching that the pure “trans” isomer for a Gemini-diene compound is required to successfully form a cubic liquid crystal phase, in particular the “trans” isomer is always used to form bicontinuous cubic phases.

### SUMMARY OF THE INVENTION

The present invention solves the limitations of the prior art by providing a diene-based Gemini polymerizable surfactant that has a near-equal mixture of the “cis” and “trans” isomers and that also can form a bicontinuous cubic phase. The new Gemini surfactant is produced by a chemical synthesis method involving fewer transformation steps. The present invention teaches the production and use of mix isomer diene Gemini polymerizable surfactants to form the bicontinuous cubic phase and to form polymers made from the same. Desalination membranes with the bicontinuous cubic phase polymer can be made using the material of the present invention.

The present invention provides a Gemini polymerizable surfactant composition having the general formula:

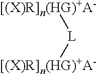

wherein, A⁻ is an anion; HG is a head group; L is a spacer group which comprises an alkyl group having from 1 to 12 carbon atoms; R is a hydrophobic tail group comprising a linear alkyl group attached to a polymerizable group X, with the tail group as a whole (the R and connected X group combined) having at least 10 carbon atoms; wherein, n is 1; wherein, the polymerizable group X is a diene group with 40 to 60 percent abundance of diene isomer “E”, and with 40 to 60 percent abundance of diene isomer “Z”; and wherein, the Gemini polymerizable surfactant forms a cubic lyotropic liquid crystalline phase. Each combined R and attached X group as a whole has at least 10 carbon atoms. Optionally, the head group is an imidazolium group and the anion is a bromide. The spacer group may optionally be a linear alkylene group with 4 to 6 carbon atoms, and R may optionally be a linear C14 alkylene group, a 010 alkylene group, while L may independently be a linear C6 alkylene group, an C4 alkylene group or a —CH2-O—CH2- group. The abundance of diene isomer “E” may be expressed as Ab(E) and the abundance of diene isomer “Z” may be expressed as Ab(Z). Ab(E) is between 0.40 and 0.60 (40% and 60%) and Ab(E)+Ab(Z)=1.00 (100%). The present invention also provides a Gemini polymerizable surfactant composition having the general formula:

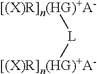

wherein, A⁻ is an anion; HG is a head group; L is a spacer group which comprises an alkyl group having from 1 to 12 carbon atoms; R is a hydrophobic tail group comprising a linear alkyl group attached to a polymerizable group X, with the tail group as a whole having at least 10 carbon atoms; wherein, n is 1; wherein, the polymerizable group X is a diene group with 40 to 60 percent abundance of diene isomer “E”, and with 40 to 60 percent abundance of diene isomer “Z”; wherein, the Gemini polymerizable surfactant forms a cubic lyotropic liquid crystalline phase, in which the Gemini polymerizable surfactant is made by a process comprising the step of: reacting an aldehyde with an allyl ylide, forming a diene compound. Optionally, the allyl ylide is a phosphorous ylide and, more preferably the phosphorous ylide is an allytriphenyl phosphonium bromide.

The present invention also provides a Gemini polymerizable surfactant composition having the general formula:

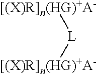

wherein, A⁻ is an anion, HG is a head group; L is a spacer group which comprises an alkyl group having from 1 to 12 carbon atoms; R is a hydrophobic tail group comprising a linear alkyl group attached to a polymerizable group X, with the tail group as a whole having at least 10 carbon atoms; wherein, n is 1; wherein, the polymerizable group X is a diene group with 40 to 60 percent abundance of diene isomer “E”, and with 40 to 60 percent abundance of diene isomer “Z”; wherein, the Gemini polymerizable surfactant forms a cubic lyotropic liquid crystalline phase, in which the Gemini polymerizable surfactant is made by a process comprising the step of: transforming an aldehyde into a diene without the use of a Peterson Elimination. Optionally, the transforming an aldehyde into a diene is performed without the use of a Matteson's Reagent.

### BRIEF DESCRIPTION OF THE DRAWINGS

FIG. 1. Preparation and chemical structure of a diene gemini polymerizable surfactant with mixed cis and trans isomers.

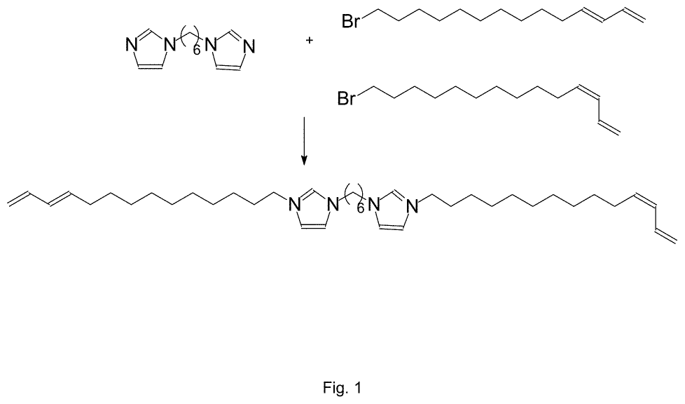

FIG. 2. Chemical structure of alkyl diene tail group precursors having “E” and “Z” conformations.

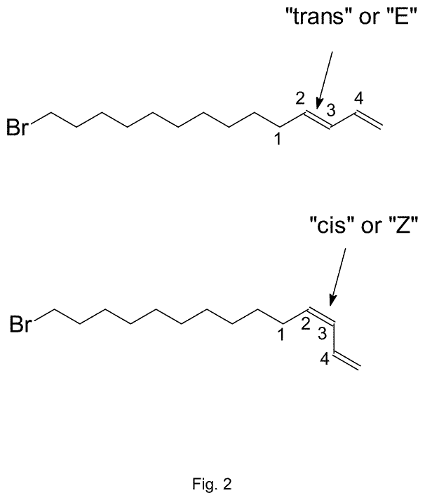

FIG. 3. X-ray diffraction spectrum for the material of Example 7, a mixed isomer Gemini polymerizable surfactant showing formation of bicontinuous cubic phase.

FIG. 4. X-ray diffraction spectrum for the material of Example 8, a 95% pure “E” isomer Gemini polymerizable surfactant showing formation of bicontinuous cubic phase.

### DETAILED DESCRIPTION OF THE INVENTION

The summary of the invention above and in the Detailed Description of the Invention, and the claims below, and in the accompanying drawings, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

The term “comprises” and grammatical equivalents thereof are used herein to mean that other components, ingredients, steps, etc. are optionally present. For example, and article “comprising” (or “which comprises”) component A, B, and C can consist of (i.e. contain only) components A, B, and C, or can contain not only components A, B, and C but also one or more other components.

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending on the variable being defined). For example, “at most 4” means 4 or less than 4, and “at most 40%” means 40% or less than 40%. When, in this specification, a range is given as “(a first number) to (a second number)” or “(a first number)-(a second number)”, this means a range whose lower limit is the first number and whose upper limit is the second number. For example 25 to 100 mm means a range whose lower limit is 25 mm, and whose upper limit is 100 mm.

The diene isomer “E”, which is also called “trans”, means the isomer in which the double bond in the diene group has a conformation in which the higher priority groups are on opposite sides of the double bond (the carbon chain and the other double bond). The term “E” comes from the first letter of the German word for opposite, or “entgengen”.

The diene isomer “Z”, which is also called “cis”, means the isomer in which the double bond in the diene group has a conformation in which the higher priority groups are on same sides of the double bond (the carbon chain and the other double bond). The term “Z” comes from the first letter of the German word for together, or “zusammen”.

A Peterson Olefination, also referred to as a Peterson Elimination, is a two-step synthesis of an olefin involving the addition of an α-silyl carbanion to a carbonyl compound to form a β-silyl alcohol and then the elimination of a silol. A Peterson Elimination reaction can use a Matteson's Reagent, which is a specific α-silyl carbanion, further defined by its chemical structure in Scheme 3.

A ylide is a molecule that bears no overall charge but has a negatively charged carbon atom bonded to a positively charged heteroatom.

A phosphorous ylide has a phosphorous atom as the positively charged heteroatom.

An ally ylide has an ally group bonded to the negatively charged carbon atom of the ylide.

Gemini polymerizable surfactants may be described by the following general structure;

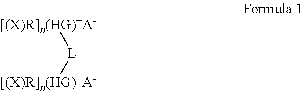

where X is a polymerizable group, R is a hydrophobic tail group having at least 8 carbon atoms optionally comprises a polymerizable group X, n=1 for a Gemini surfactant with two head groups and two tail groups (1 tail group per each headgroup), and L is a spacer or linkage group. “HG” represents a headgroup that may be cationic, and may be a phosphonium group, an imidazolium, or other cationic group. These compounds are commonly referred to as “Gemini” surfactants due to the presence of two head groups. The “A⁻” in the structure can be any suitable anion group (for example bromide).

In an embodiment, X, R, n and L may be as described above. In another embodiment, the LLC salt surfactant may be described as the Gemini surfactant wherein X may be either an acrylate, methacrylate or diene polymerizable group; R may be an alkyl chain containing from 8 to 20 carbon atoms, n may be 1, the cationic headgroup (HG) may be either a phosphonium or imidazolium group; and the anion (A⁻) may be a chloride, bromide, trifluoromethane sulfonate, para-toluene sulfonate or perchlorate group. A critical difference between the prior art and the present invention is that synthesis of the material of the present invention does not rely on the use of a Matteson's reagent nor does it produce dienes that are highly enriched in one isomer. This significant improvement is found in the preparation of the tail groups prior to addition to the head groups.

For example, in the more lengthy synthesis of the materials, Hoag and Gin (Macromolecules Vol. 33 No. 23, 2000) teach the following synthetic preparation of the tail group precursors (Scheme 1).

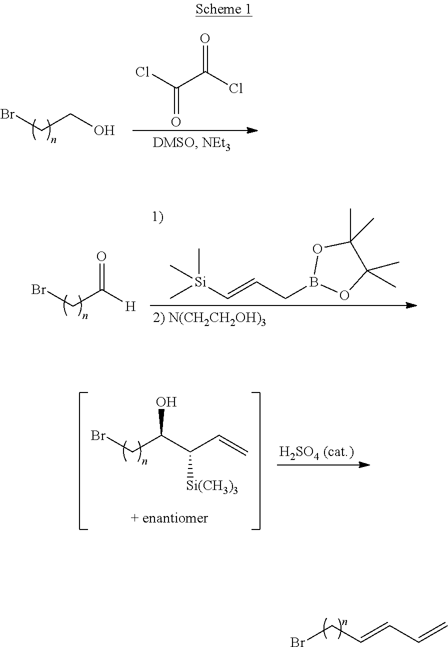

In Scheme 1, the deoxysilylation under acidic conditions (Peterson elimination) affords the desired bromo-1,3-diene tail unit with a good yield and an E:Z ratio of 20:1 (or 95.2% E: at Hoag et al.). If the Peterson elimination is conducted under basic conditions the product is 10:1 Z;E (or 90.9% Z). The bromoalkyl-aldehyde may be produced by other means, but the above scheme requires the Matteson's reagent.

The lengthy preparation of Matteson's Reagent is as follows in Scheme 2 and Scheme 3.

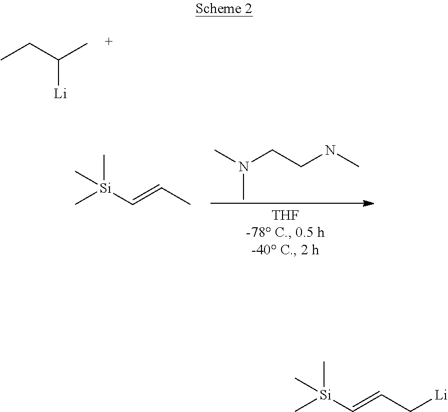

All glassware was heated in an oven at 110° C. for 2 hours, and all anhydrous solvents were further dried with molecular sieves and purged with argon for 10 min, prior to the synthesis. To a 500 mL 3-neck round bottom flask equipped with a stir bar and an addition funnel, a solution of N,N,N′,N′-tetramethylethylenediamine (19.6 mL, 0.13 mol) in 80 mL of anhydrous THF was cooled to −78° C. under argon purge with an acetone/dry ice bath. Then, 1.3M sec-BuLi solution (100 mL, 0.13 mol) was added to the solution. Allyltrimethylsilane (20.7 mL, 0.13 mol) was mixed with 20 mL of anhydrous THF and added dropwise to the flask from the addition funnel. The temperature was kept at −78° C. for 30 min. Then, the temperature was slowly raised to but not higher than −40° C. in the course of 2 hours by controlling the amount of dry ice in the acetone bath. The next step is shown in Scheme 3.

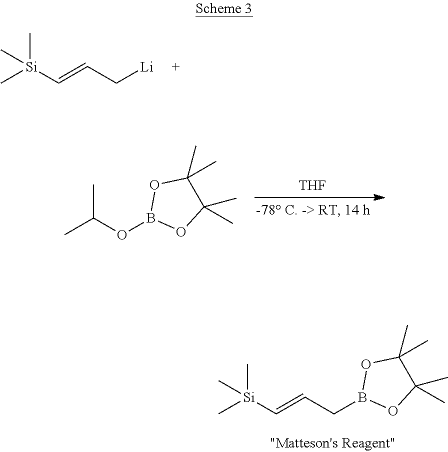

To another 1000 mL 3-neck round bottom flask equipped with a mechanical stir bar, a solution of 2-isopropoxy-4,4,5,5-tetramethyl-1,3,2-dioxaborolane (26.5 mL, 0.13 mol) in 60 mL of anhydrous THF was cooled to −78° C. under argon purge with an acetone/dry ice bath. The lithiated solution was then transferred to the 1000 mL flask through a cannula. The mixture was allowed to warm up to room temperature and stirred for 14 hours. The clear product solution was next added to a mixture of saturated aqueous NH₄Cl (150 mL), 1M HCl (150 mL), and CH₂Cl₂ (300 mL). The mixture was extracted with diethyl ether (300 mL), and the organic fraction was washed with H₂O (3×150 mL) and brine, NaCl and water (1×100 mL). The organic fraction was dried with MgSO₄, filtered, and the solvent was evaporated in vacuum at about 40° C. to afford the crude product. After fractional distillation under reduced pressure at 75° C., the pure product was collected as a clear colorless liquid.

An embodiment of the present invention is a synthesis method that produces the diene tail group precursors without relying on the use of a Matteson's reagent (as described above), and which produces mixed isomers of the E and Z conformation. The present invention teaches the following simplified synthetic preparation of the tail group precursors (Scheme 4).

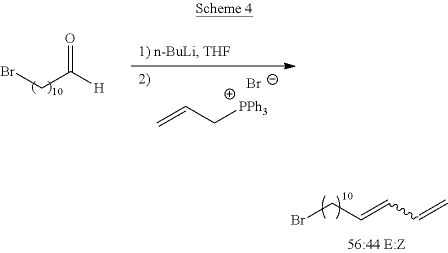

In Scheme 4, a non-limiting example of a 14-carbon tail is described; however, other embodiments of the present invention include analogous compounds having a minimum of 8 carbons in the tail group. The synthesis of the exemplary 14-bromo-tetradeca-1,3-diene is as follows.

Allyltriphenylphosphonium bromide (4.5 g, 12 mmol) and tetrahydrofuran (100 mL) were combined in an oven-dried 250 mL round-bottom flask, equipped with stir bar, in an argon atmosphere. The round-bottom flask was immersed in an ice bath. n-Butyllithium (7.5 mL, 1.6 M, 12 mmol) was added dropwise. The resulting orange solution was aged at 0° C. for 0.5 hours before adding 10-bromodecanal (2.5 g, 10 mmol) in tetrahydrofuran (2 mL) dropwise. The reaction mixture was allowed to warm to ambient temperature over 18 hours. The resulting colorless solution was quenched with saturated NH₄Cl solution (20 mL) and the resulting layers separated. The aqueous layer was extracted with diethyl ether (2×100 mL). The combined organic layers were dried over MgSO₄ and the solvents removed in vacuo. The resulting yellow residue was tritrated with warm diethyl ether (10 mL) and layered with hexanes (10 mL). The resulting phases were allowed to slowly mix for 2.5 hours. The resulting slurry was filtered and the solvents removed in vacuo. The resulting white residue was filtered through a plug of basic activity I alumina, eluting with hexanes, to yield a pale yellow oil (0.9 g, 35%). The final product had a 56:44 ratio of the conformations (E:Z).

A non-limiting example headgroup preparation follows in Scheme 5.

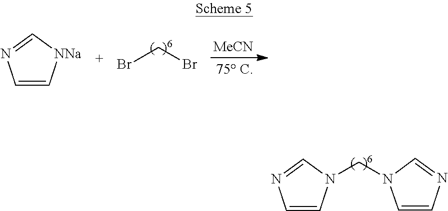

Sodium imidazolate (9.0 g, 100 mmol) and acetonitrile (50 mL) were combined in a 100 mL round-bottom flask, equipped with stir bar, in an argon atmosphere. The round-bottom flask was immersed in an ambient temperature water bath. 1,6-dibromohexane (7.7 mL, 50 mmol) was added dropwise to the resulting slurry by syringe. The reaction mixture was allowed to stir at ambient temperature for 0.5 hours before heating to 75° C. for 20 hours. The resulting slurry was cooled to ambient temperature and the solvent was removed in vacuo. 50 mL Dichloromethane and 25 mL water were added and the resulting layers separated. The aqueous layer was further extracted with dichloromethane (2×50 mL). The combined organic layers were dried over MgSO₄ and the solvents removed in vacuo. The resulting white crystalline solid was recrystallized from warm ethyl acetate to yield a white crystalline solid (5.8 g. 53%). Analogous headgroups with different spacer groups or linkers are possible, for example a 4-carbon spacer group, a diethylether group and the like.

The Gemini surfactants were made next from either the tail precursors of Scheme 1 or Scheme 4 and the headgroup precursor of Scheme 5 in the following final step of the synthesis (Scheme 6).

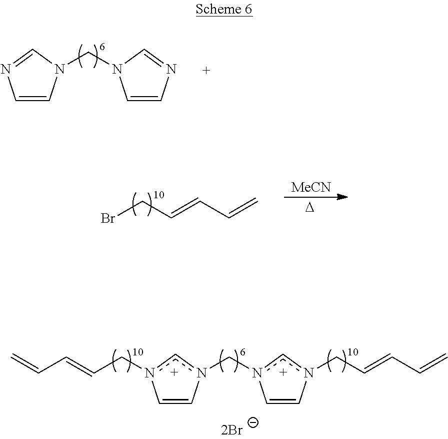

1,1-(1,6-Hexanediyl)bisimidazole (1.07 g, 4.89 mmol, 100 mol %) was dissolved in acetonitrile (15 mL) in a 100-mL round-bottom flask equipped with a stir bar and reflux condenser. Addition of 14-bromotetradeca-1,3-diene (2.70 g, 9.90 mmol, 203 mol %) produced a clear, light yellow solution that was stirred at 86° C. for 96 hours. Upon cooling to room temperature, solvent was concentrated under rotary vacuum to afford an off-white, waxy solid that was stirred in hexanes (3×50 mL) and filtered to afford the product as a white, crystalline solid. Yield: 3.1 g (82%).

However, the chemical structure in Scheme 6 shows both dienes as the trans isomer. For the improved materials of the present invention there is a near-equal mixture of the cis and trans isomer, as represented in Scheme 7.

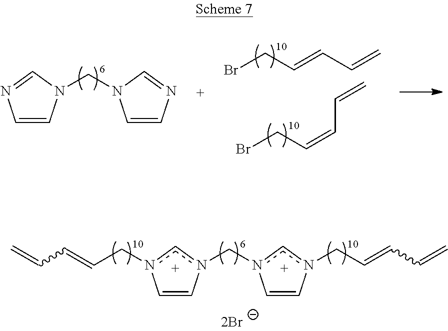

### EXAMPLE 1

Synthesis of Gemini 18EZ-6, with two imidazolium head groups and mixed isomer diene polymerizable groups. The preparation of the C18 tail precursor is as follows in Scheme 8 through Scheme 11.

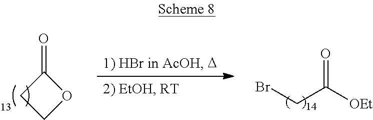

ω-Pentadecalactone (12 g, 50.0 mmol) and hydrobromic acid (36 mL, 33 wt % in AcOH) were combined in a 50 mL Schlenk tube. The reaction mixture was heated to 120° C. for 3 days. While the reaction mixture was still warm, it was poured into a round-bottom flask containing absolute ethanol (30 mL), and equipped with a stir bar. The reaction mixture was allowed to stir at ambient temperature for 18 hours. The ethanol was removed in vacuo, and ethyl acetate (100 mL) was added to the resulting off-white solid. The resulting layers were separated, and the organic layer was washed successively with saturated sodium bicarbonate (2×50 mL) and brine (50 mL). The organic layer was dried over MgSO₄ and the solvents were removed in vacuo. The resulting off-white solid was filtered through a plug of silica (20:1 v/v hexanes:ethyl acetate) to yield a white powder (14 g, 80%).

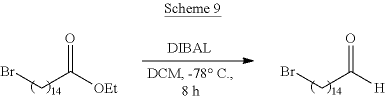

15-Bromo-ethyl-pentadecanoate (1.75 g, 5.01 mmol) was dissolved in CH₂Cl₂ (15 mL) in an oven-dried 50 mL round-bottom flask, equipped with stir bar, in an Ar atmosphere. The resulting solution was cooled in a dry-ice/acetone bath. The reaction mixture became turbid. Diisobutylaluminium hydride (5.5 mL, 1 M in CH₂Cl₂, 5.5 mmol) was added dropwise. The resulting solution was aged 8 hours before the reaction was quenched with methanol (5 mL) and allowed to warm to ambient temperature. The reaction mixture was poured into a stirring solution of 1.2 M HCl (100 mL). The resulting biphasic mixture was stirred for 45 mins at ambient temperature before the separating the layers. The aqueous layer was further extracted with CH₂Cl₂ (3×25 mL). The solvents were removed in vacuo to yield a white powder (1.46 g, 95%).

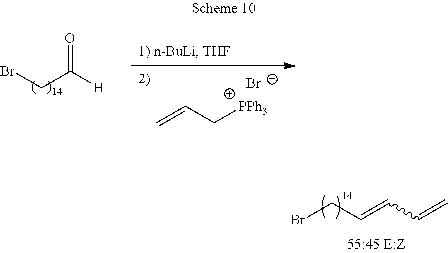

Allyltriphenylphosphonium bromide (10.3 g, 26.8 mmol) and tetrahydrofuran (200 mL) were combined in an oven-dried 250 mL round-bottom flask, equipped with stir bar, in an argon atmosphere. The round-bottom flask was immersed in an ice bath. n-Butyllithium (17.0 mL, 1.6 M, 26.8 mmol) was added dropwise. The resulting orange solution was aged at 0° C. for 0.5 h before adding 15-bromopentadecanal (6.83 g, 22.4 mmol) in tetrahydrofuran (5 mL) dropwise. The reaction mixture was allowed to warm to ambient temperature over 18 hours. The resulting colorless solution was quenched with saturated NH₄Cl solution (40 mL) and the resulting layers separated. The aqueous layer was extracted with ethyl acetate (2×100 mL). The combined organic layers were dried over MgSO₄ and the solvents removed in vacuo. The resulting yellow residue was tritrated with warm diethyl ether (25 mL) and layered with hexanes (25 mL). The resulting phases were allowed to slowly mix for 2.5 hours. The resulting slurry was filtered and the solvents removed in vacuo. The resulting white residue was filtered through a plug of basic alumina, eluting with hexanes, to yield a colorless oil (3.17 g, 43%). ¹H NMR (500 MHz, CDCl₃-c) δ 6.63 (m, 0.45H), 6.30 (m, 0.55H), 6.01 (m, 1H), 5.70 (m, 0.55H), 5.45 (m, 0.45H), 5.21-4.89 (m, 2H), 3.40 (m, 2H), 2.21-2.13 (m, 0.9H), 2.06 (m, 1.1H), 1.84 (m, 2H), 1.46-1.32 (m, 4H), 1.26 (m, 18H). The final product had a 55:45 ration of the E:Z conformations as determined by proton NMR.

The 18EZ-6 Gemini surfactant is made next from the tail precursor of Scheme 10 and the headgroup precursor of Scheme 5 in the following final step of the synthesis (Scheme 11).

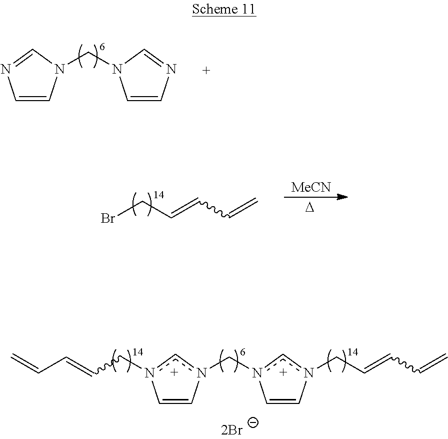

1,1-(1,6-Hexanediyl)bisimidazole (327 mg, 1.50 mmol) and 18-bromooctadeca-1,3-diene (1.09 g, 3.3 mmol) were dissolved in acetonitrile (6 mL) in a 25-mL round-bottom flask equipped with a stir bar and reflux condenser. The resulting solution was heated to reflux for 96 hours. Upon cooling to ambient temperature, the solvent was concentrated in vacuo and the product precipitated from 125 mL cold, stirring diethyl ether to afford the product as a white powder (1.15 g, 88%).

### EXAMPLE 2

Synthesis of Gemini 18EZ-4, with two imidazolium head groups and mixed isomer diene polymerizable groups. The preparation of the 1,1-(1,4-Butanediyl)bisimidazole headgroup precursor is as follows in Scheme 12.

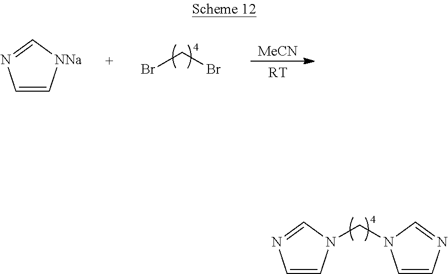

Sodium imidazolate (9.0 g, 100 mmol) and acetonitrile (50 mL) were combined in an oven-dried 100 mL round-bottom flask, equipped with stir bar, in an argon atmosphere. The round-bottom flask was immersed in an ambient temperature water bath. 1,4-dibromobutane (5.1 mL, 50 mmol) was added dropwise to the resulting slurry by syringe. The reaction mixture was allowed to stir at ambient temperature for 24 hours. The acetonitrile was removed in vacuo. 50 mL Dichloromethane and 25 mL water were added and the resulting layers separated. The aqueous layer was further extracted with dichloromethane (2×50 mL). The combined organic layers were dried over MgSO₄ and the solvents removed in vacuo. The resulting white crystalline solid was recrystallized from warm ethyl acetate to yield a white crystalline solid (3.9 g. 41%).

The 18EZ-4 Gemini surfactant is made from the tail precursor of Scheme 10 and the headgroup precursor of Scheme 13 in the following final step of the synthesis (Scheme 13).

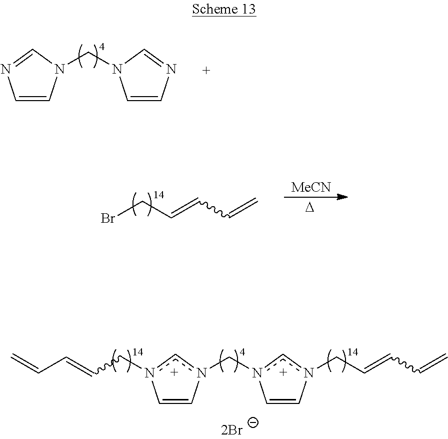

1,1-(1,4-Butanediyl)bisimidazole (148 mg, 0.78 mmol) and 18-bromooctadeca-1,3-diene (563 mg, 1.71 mmol) were dissolved in acetonitrile (3 mL) in a 25-mL round-bottom flask equipped with a stir bar and reflux condenser. The resulting solution was heated to reflux for 96 hours. Upon cooling to ambient temperature, the solvent was concentrated in vacuo and the product precipitated from 75 mL cold, stirring diethyl ether to afford the product as a white powder (552 mg, 84%).

### EXAMPLE 3

Synthesis of Gemini 14EZ-4, with two imidazolium head groups and mixed isomer diene polymerizable groups. The preparation of the C14 tail precursor is as follows in Scheme 14.

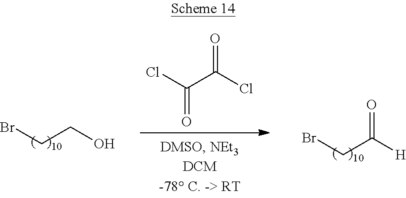

Oxalyl chloride (2.0 mL, 24 mmol) was dissolved in dichloromethane (80 mL) in an oven-dried 250-mL round-bottom flask, equipped with stir bar, in an argon atmosphere. The reaction mixture was cooled in a dry ice/acetone bath, before adding dimethyl sulfoxide (3.4 mL, 48 mmol) in dichloromethane (10 mL), dropwise. The reaction mixture was aged 5 mins before adding bromoundecanol (5.0 g, 20 mmol) in dichloromethane (20 mL), dropwise. The reaction mixture was aged 5 mins before adding triethylamine (13.9 mL, 100 mmol), dropwise. The reaction mixture was aged 10 mins before the bath was removed, and the reaction mixture allowed to warm slowly to ambient temperature. Dichloromethane (50 mL) and deionized water (50 mL) were added and the resulting layers separated. The aqueous layer was extracted with dichloromethane (50 mL). The combined organic layers were washed with 1.2 M HCl (2×50 mL) and brine (50 mL). The organic layer was dried over MgSO₄ and the solvents removed in vacuo. The resulting yellow oil was filtered through a plug of silica, eluting with 10:1 v/v hexanes:ethyl acetate to afford the product as a white, waxy solid (4.57g, 92%).

The 14EZ-6 Gemini surfactant is made from the tail precursor of Scheme 4 and the headgroup precursor of Scheme 5 in the following final step of the synthesis (Scheme 15).

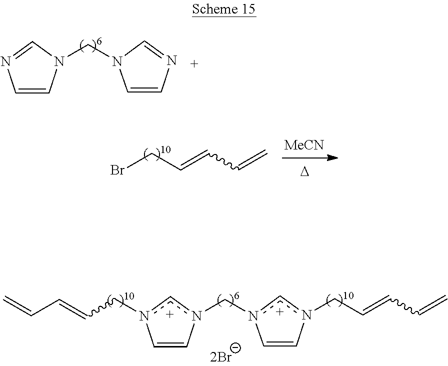

1,1-(1,6-Hexananediyl)bisimidazole (218 mg, 1 mmol) and 14-bromotetradeca-1,3-diene (600 mg, 2.2 mmol) were dissolved in acetonitrile (4 mL) in a 25-mL round-bottom flask equipped with a stir bar and reflux condenser. The resulting solution was heated to reflux for 96 h. Upon cooling to ambient temperature, the solvent was concentrated in vacuo and the product precipitated from 75 mL cold, stirring diethyl ether to afford the product as a white crystalline solid (622 mg, 81%).

### EXAMPLE 3

Synthesis of Gemini 14EZ-4, with two imidazolium head groups and mixed isomer diene polymerizable groups. The 14EZ-4 Gemini surfactant is made from the tail precursor of Scheme 4 and the headgroup precursor of Scheme 12 in the following final step of the synthesis (Scheme 16).

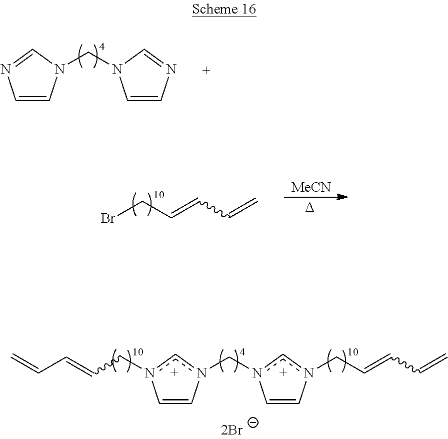

1,1-(1,4-Butanediyl)bisimidazole (190 mg, 1 mmol) and 14-bromotetradeda-1,3-diene (600 mg, 2.2 mmol) were dissolved in acetonitrile (4 mL) in a 25-mL round-bottom flask equipped with a stir bar and reflux condenser. The resulting solution was heated to reflux for 96 h. Upon cooling to ambient temperature, the solvent was concentrated in vacuo and the product precipitated from 75 mL cold, stirring diethyl ether to afford the product as a white crystalline solid (710 mg, 96%).

### EXAMPLE 4

Alternative synthesis of Gemini 18EZ-6 with two imidazolium head groups and mixed isomer diene polymerizable groups. The alternate final step of the preparation of the C18 tail precursor is as follows in Scheme 17.

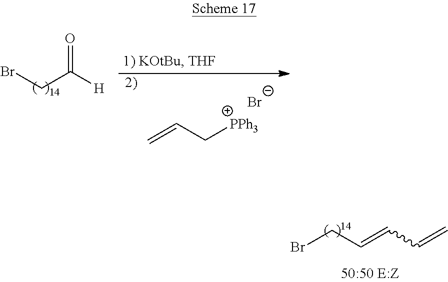

Allyltriphenylphosphonium bromide (10.0 g, 26.0 mmol) and tetrahydrofuran (100 mL) were combined in a 250 mL round-bottom flask. Potassium tert-butoxide (3.69 g, 24.0 mmol) was added portion-wise. The resulting red-orange solution was aged at ambient temperature for 2 hours before adding 15-bromopentadecanal (6.1 g, 20.0 mmol) in tetrahydrofuran (5mL) dropwise. The reaction mixture was allowed to stir at ambient temperature over 18 hours. The reaction mixture was quenched with saturated NH₄Cl solution (40 mL) and the resulting layers separated. The aqueous layer was extracted with ethyl acetate (2×100 mL). The combined organic layers were dried over MgSO₄ and the solvents removed in vacuo. The resulting orange residue was tritrated with warm diethyl ether (25 mL) and layered with hexanes (25 mL). The resulting phases were allowed to slowly mix for 2.5 hours. The resulting slurry was filtered and the solvents removed in vacuo. The resulting yellow residue was filtered through a plug of neutral alumina, eluting with hexanes, to yield a colorless oil. The final product had a 55:45 E:Z conformation as determined by proton NMR.

The 18EZ-6 Gemini surfactant is made next from the tail precursor of Scheme 10 and the headgroup precursor of Scheme 5 in the following final step of the synthesis (Scheme 18).

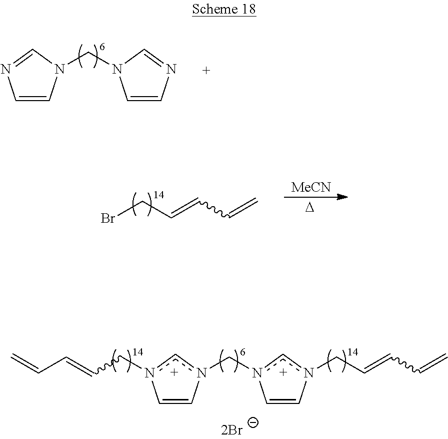

1,1-(1,6-Hexanediyl)bisimidazole (327 mg, 1.50 mmol) and 18-bromooctadeda-1,3-diene (1.09 g, 3.3 mmol) were dissolved in acetonitrile (6 mL) in a 25-mL round-bottom flask equipped with a stir bar and reflux condenser. The resulting solution was heated to reflux for 96 hours. Upon cooling to ambient temperature, the solvent was concentrated in vacuo and the product precipitated from 125 mL cold, stirring diethyl ether to afford the product as a white powder.

### EXAMPLE 5

Preparation of desalination membrane samples. Membranes prepared for use in XRD and desalination studies were prepared following this procedure. A porous polysulfone ultra filtration membrane was used as a support. This type of membrane is commercially used for clarifying food and beverages. The membrane had a molecular weight cutoff of 20 kDa and a pure water flux of 1530 lmh/bar. The support was cleaned by soaking a 6-inch by 10-inch piece of the support in methanol for 30 minutes, then air dried for 15 minutes. It was then affixed to a flat, rigid metal plate.

Separately, the casting solution was prepared. The solution was 90 wt % methanol and 10 wt % of a the mixture: [79.7/19.8/0.6 (wt %/wt %/wt %) of a Gemini polymerizable surfactant/water/2-Hydroxy-2-methylpropiophenone (2-HMP)]. 2-HMP is a photoinitiator. This solution was cast onto the mounted membrane support using an automatic roll caster with a ⅜″ diameter rod (#3 wire) at a casting speed of 9 inches per second. Next the membrane was allowed to dry (evaporate methanol). The next step was to rotate the membrane 180 degrees and cast another layer of the solution and let it air dry for 15 minutes. The membrane was next annealed in air and remove any remaining methanol by raising the temp slowly to 75° C. then set aside to cool. The membrane is next exposed to an argon purge, with an O₂%<2% and heated back to 75° C. and photopolymerized with UV light (10 mW/cm²) for 1 hour.

### EXAMPLE 6

Synthesis of Gemini 18E-6 with two imidazolium head groups and mixed isomer diene polymerizable groups. The preparation of the C18 tail precursor is as follows in Scheme 19 through Scheme 22.

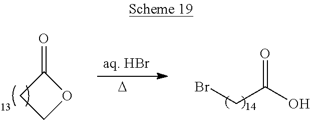

ω-Pentadecalactone (20.69 g, 86.09 mmol, 100 mol %) was stirred in 48% HBr (98 mL, 1.076 mmol, 1250 mol %) in a 250-mL round-bottom flask equipped with a stir bar and reflux condenser. H₂SO₄ (13 mL, 243.88 mmol, 283 mol %) was added dropwise to minimize exothermic activity, liquefying the solid lactone. The yellow emulsion was heated to 115° C. and stirred for 40 hours. The resultant brown solution was extracted into CHCl₃ (200 mL), washed with de-ionized H₂O (3×100 mL) and brine (3×100 mL), dried over anhydrous MgSO₄. The resulting organic solution was reduced under rotary vacuum to afford a light yellow solid, which was then recrystallized from hot CHCl₃ and washed with cold hexanes to afford the product as a white, crystalline solid. Yield: 20.9 g (76%).

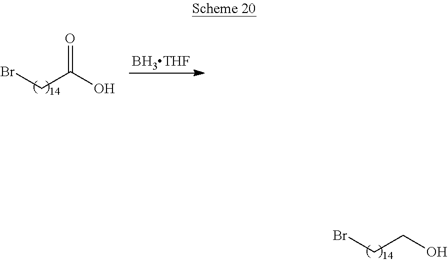

Borane-THF complex solution (83 mL, 83 mmol, 210 mol %) was measured out into a 500-mL Schlenk flask equipped with a stir bar. 15-Bromo-1-pentadecanoic acid (12.70 g, 39.53 mmol, 100 mol %) was added slowly to minimize bubbling, and the light yellow solution turned clear over the course of 40 hours of stirring. The reaction solution was then quenched with DI H₂O (10 mL) dropwise, extracted into Et₂O (50 mL), and washed with de-ionized H₂O (3×50 mL) and brine (3×50 mL), dried over anhydrous MgSO₄, and evaporated to afford the product as a white solid. Yield: 39.4 g (99%).

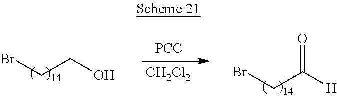

15-Bromopentanol (6.69 g, 21.78 mmol, 100 mol %) was dissolved in CH₂Cl₂ (200 mL) in a 500-mL round-bottom flask equipped with a stir bar. To the clear, slightly yellow solution was added PCC on alumina (39.76 g, 37.48 mmol, 172 mol %) with vigorous stirring. The slurry was stirred at room temperature for 40 hours. Reduction of the CH₂Cl₂ via rotary vacuum produced a dark brown solid that was stirred in diethyl ether and filtered through a pad of SiO₂, washing with diethyl ether (700 mL). Concentration of the diethyl ether under rotary evaporation afforded the product as a white solid. Yield: 21.1 g (92%).

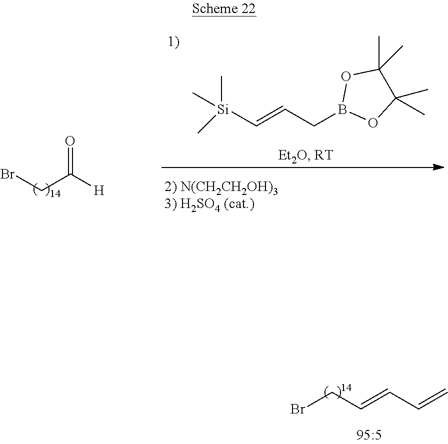

15-Bromopentanal (6.42 g, 21.0 mmol) was dissolved in diethyl ether (50 mL). To this solution was added Matteson's reagent (6.06 g, 25.2 mmol). The reaction was stirred for 60 h before adding triethanolamine (4.70g, 31.5 mmol) and stirring for an additional 6 h. During this time a white precipitate formed. The reaction mixture was washed with saturated NaHCO₃ (3×125 mL) and brine (2×25 mL), dried over MgSO₄, concentrated and dried in vacuo. The resulting oil was purified by passage through a silica plug with hexanes (150 mL) first (discarded) and then CH₂Cl₂ (400 mL).The resulting oil was dissolved in diethyl ether (125 mL) and concentrated H2SO₄ (13 drops) was added; the solution was stirred for 48 hours. The reaction mixture was diluted with hexanes, washed with saturated aqueous NaHCO3 (3×125 mL), dried over MgSO4, concentrated and dried in vacuo. The crude oil was purified by column chromatography on silica, eluting with hexanes to give 4.25 g (61.4%) of the pure product as a clear, colorless oil.

The 18E-4 Gemini surfactant is made from the tail precursor of Scheme 22 and the headgroup precursor of Scheme 5 in the following final step of the synthesis (Scheme 23).

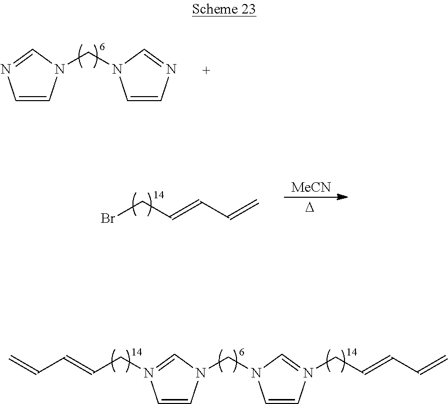

18-Bromooctadeca-1,3-diene (4.25 g, 12.90 mmol, 204 mol %) and 1,1′-(1,6-hexanediyl)bisimidazole (1.38 g, 6.32 mmol, 100 mol %) were dissolved in acetonitrile (70 mL) in a 250-mL round-bottom flask equipped with a stir bar and reflux condenser. The clear, light yellow solution was stirred at 84° C. for 100 hours. Concentration of the reaction solvent via rotary evaporation produced an off-white solid, which was stirred in hexanes (3×200 mL) and filtered to afford the product as a white, crystalline solid. Yield: 5.2 g (93%).

### EXAMPLE 7

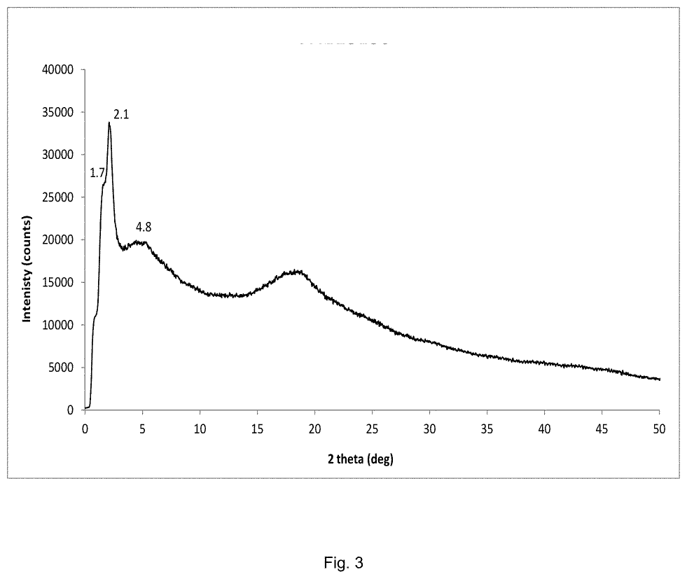

XRD of mixed isomer Gemini polymerizable surfactant showing formation of bicontinuous cubic phase: The membrane preparation method of Example 5 was used with the Gemini polymerizable surfactant from Example 1 (Gemini 18-EZ6). A thin layer of the casted membrane was then removed, folded to increase the diffraction resolution, and analyzed by Power X-ray Diffraction (PXRD). FIG. 3 shows the x-ray diffraction spectrum for this material. The obtained diffraction spectrum had resolved peaks at 1.7°, 2.1°, and 4.8° (2-theta), a pattern which indexes to a ratio of 1/√4, 1/√6, and 1/√32 for d-spacing peaks and is characteristic of a QI phase, or a Type-I Bicontinuous Cubic Phase.

### COMPARATIVE EXAMPLE 8

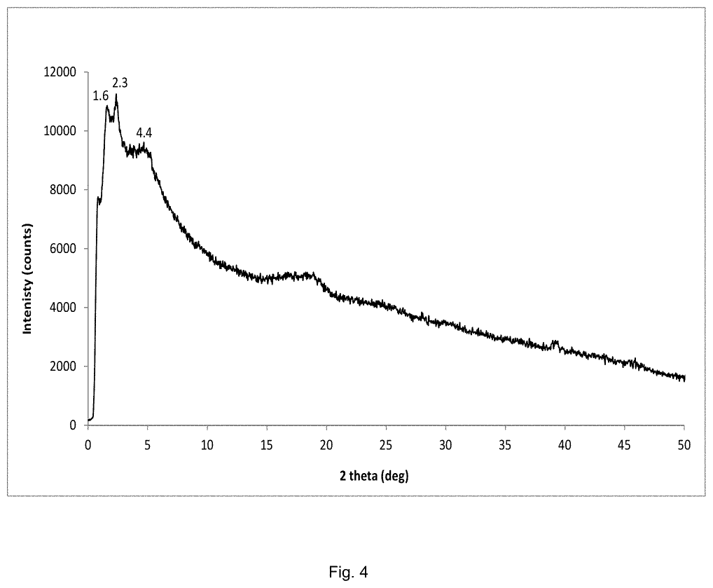

XRD of the 95% trans isomer Gemini surfactant showing formation of a bicontinuous cubic phase. The membrane preparation method of Example 5 was used with the Gemini polymerizable surfactant from Example 6 (Gemini 18E-6). A thin layer of the casted membrane was then removed, folded to increase the Q_{I} polymer active volume, and analyzed by Power X-ray Diffraction (PXRD). FIG. 4 shows the x-ray diffraction spectrum for this material. The obtained diffraction spectrum had resolved peaks at 1.6° , 2.3° , and 4.4° index to 1/√3, 1/√6, and 1/√22 for d-spacing peaks and is characteristic of a QI phase, or a Type-I Bicontinuous Cubic Phase.

Both FIG. 3 and FIG. 4 show x-ray diffraction patterns indicative of the cubic phase, but with slightly varying crystal spacing. Without wishing to be bound by theory, the x-ray diffraction pattern for the mixed isomer dienes (Example 7) indicates that that pores are likely slightly smaller than the pores formed by the material made from the 95% pure trans isomer. As shown below in Example 9, this smaller pore size results in a higher salt rejection when the membranes are used for saltwater desalination.

### EXAMPLE 9

Desalination membrane: Two types of desalination membranes made using the methods of Example 5 (Having near 50% abundance of each the “E” and “Z” conformations). One membrane was made using the Gemini surfactant from Example 1 and the other membrane was made using the Gemini surfactant of Example 6 (having 95% “E” conformation).

A stock solution of 3,000 ppm sodium chloride in deionized water was used in these tests. 200 mL were added to high-pressure dead end filtration cells fitted with the membranes. The pressure was held at 400 psi. The NaCl rejection for the Gemini surfactant made by the methods of Example 6 (the 95% “E” conformation version called Gemini 18E-6) was 97.7±0.0089% rejection at a flux of 2.9±0.0167 L*m⁻²*h⁻¹. The sodium chloride rejection and flux were both higher for the membrane made using the improved Gemini surfactant of the present invention (the mixed isomer version called Gemini 18EZ-6). The sodium chloride rejection was 99.1±0.0052% rejection at a flux of 4.5±0.1433 L*m⁻²*h⁻¹.

Although the present invention has been described in considerable detail with reference to certain preferred versions thereof, other versions are possible. Therefore, the spirit and scope of the appended claims should not be limited to the description of the preferred versions contained herein, except where required by 35 U.S.C.§ 112 ¶6 or 35 U.S.C.§ 112 (f).

The reader's attention is directed to all references which are filed concurrently with this specification and which are incorporated herein by reference.

All the features in this specification (including any accompanying claims, abstract, and drawings) may be replaced by alternative features serving the same, equivalent or similar purpose, unless expressly stated otherwise. Thus, unless expressly stated otherwise, each feature disclosed in one example only of a generic series of equivalent of similar features.

What is claimed is:

1. A Gemini polymerizable surfactant composition having the general formula: 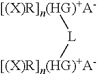
wherein,
A⁻ is an anion;
HG is a head group;
L is a spacer group which comprises an alkyl group having from 1 to 12 carbon atoms;
R is a hydrophobic tail group comprising a linear alkyl group attached to a polymerizable group X, with each combined R and attached X group as a whole having at least 10 carbon atoms;
wherein, n is 1;
wherein, the polymerizable group X is a diene group with 40 to 60 percent abundance of diene isomer “E”, and with 40 to 60 percent abundance of diene isomer “Z”; and
wherein, the Gemini polymerizable surfactant forms a cubic lyotropic liquid crystalline phase.

2. The Gemini polymerizable surfactant composition of claim 1, wherein HG is an imidazolium group and A⁻ is a bromide.

3. The Gemini polymerizable surfactant composition of claim 2, wherein L is a linear alkylene group with 4 to 6 carbon atoms.

4. The Gemini polymerizable surfactant composition of claim 2, wherein R is a linear C14 alkylene group and L is a linear C6 alkylene group.

5. The Gemini polymerizable surfactant composition of claim 2, wherein R is a linear 010 alkylene group and L is a linear C6 alkylene group.

6. The Gemini polymerizable surfactant composition of claim 2, wherein R is a linear C10 alkylene group and L is a linear C4 alkylene group.

7. A Gemini polymerizable surfactant composition having the general formula: 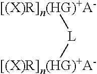
wherein,
A⁻ is an anion;
HG is a head group;
L is a spacer group which comprises an alkyl group having from 1 to 12 carbon atoms;
R is a hydrophobic tail group comprising a linear alkyl group attached to a polymerizable group X, with each combined R and attached X group as a whole having at least 10 carbon atoms;
wherein, n is 1;
wherein, the polymerizable group X is a diene group with 40 to 60 percent abundance of diene isomer “E”, and with 40 to 60 percent abundance of diene isomer “Z”;
wherein, the Gemini polymerizable surfactant forms a cubic lyotropic liquid crystalline phase; and,
wherein the Gemini polymerizable surfactant is made by a process comprising the step of: reacting an aldehyde with an allyl ylide, forming a diene compound.

8. The Gemini polymerizable surfactant composition of claim 7, wherein the step of reacting an aldehyde with an allyl ylide, forming a diene compound, is a phosphorous ylide.

9. The Gemini polymerizable surfactant composition of claim 8, wherein the phosphorous ylide is an allytriphenyl phosphonium bromide.

10. A Gemini polymerizable surfactant composition having the general formula: 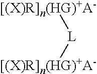
wherein,
A⁻ is an anion;
HG is a head group;
L is a spacer group which comprises an alkyl group having from 1 to 12 carbon atoms;
R is a hydrophobic tail group comprising a linear alkyl group attached to a polymerizable group X, with each combined R and attached X group as a whole having at least 10 carbon atoms;
wherein, n is 1;
wherein, the polymerizable group X is a diene group with 40 to 60 percent abundance of diene isomer “E”, and with 40 to 60 percent abundance of diene isomer “Z”;
wherein, the Gemini polymerizable surfactant forms a cubic lyotropic liquid crystalline phase; and
wherein, the Gemini polymerizable surfactant is made by a process comprising the step of: transforming an aldehyde into a diene without use of a Peterson Elimination.

11. The Gemini polymerizable surfactant composition of claim 10, further comprising the step of:
transforming an aldehyde into a diene without use of a Matteson's Reagent.
